SEQUENCE LISTING (24 sequences)

SEQ ID NO: 1 - protein, 152 aa, Artificial Sequence
  The sequence is synthesized.
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 2 - DNA, 456 bp, Artificial Sequence
  The sequence is synthesized.
cgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgag

SEQ ID NO: 3 - protein, 152 aa, Artificial Sequence
  The sequence is synthesized.
RPSGRKSSKMQAFRIWDVNQKTFYLRNNQLVAGYLQGPNVNLEEKIDVVPIEPHALFLGIHGGKMCLSCVKSGNETRLQLEAVNITDLSENRKQDKRFAFIRSDSGPTTSFESAACPGWFLCTAMEADQPVSLTNMPDEGVMVTKFYFQEDE

SEQ ID NO: 4 - DNA, 456 bp, Artificial Sequence
  The sequence is synthesized.
cgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtaatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgag

SEQ ID NO: 5 - protein, 152 aa, Artificial Sequence
  The sequence is synthesized.
RPSGTKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 6 - DNA, 456 bp, Artificial Sequence
  The sequence is synthesized.
cgaccctctgggacaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgag

SEQ ID NO: 7 - protein, 152 aa, Artificial Sequence
  The sequence is synthesized.
RPSGRKSSKMQAFTIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 8 - DNA, 456 bp, Artificial Sequence
  The sequence is synthesized.
cgaccctctgggagaaaatccagcaagatgcaagccttcacaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgag

SEQ ID NO: 9 - protein, 152 aa, Artificial Sequence
  The sequence is synthesized.
RPSGTKSSKMQAFTIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 10 - DNA, 456 bp, Artificial Sequence
  The sequence is synthesized.
cgaccctctgggacaaaatccagcaagatgcaagccttcacaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgag

SEQ ID NO: 11 - protein, 152 aa, Artificial Sequence
  The sequence is synthesized.
RPSGTKSSKMQAFRIWDVNQKTFYLRNNQLVAGYLQGPNVNLEEKIDVVPIEPHALFLGIHGGKMCLSCVKSGNETRLQLEAVNITDLSENRKQDKRFAFIRSDSGPTTSFESAACPGWFLCTAMEADQPVSLTNMPDEGVMVTKFYFQEDE

SEQ ID NO: 12 - DNA, 456 bp, Artificial Sequence
  The sequence is synthesized.
cgaccctctgggacaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtaatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgag

SEQ ID NO: 13 - protein, 152 aa, Artificial Sequence
  The sequence is synthesized.
RPSGRKSSKMQAFTIWDVNQKTFYLRNNQLVAGYLQGPNVNLEEKIDVVPIEPHALFLGIHGGKMCLSCVKSGNETRLQLEAVNITDLSENRKQDKRFAFIRSDSGPTTSFESAACPGWFLCTAMEADQPVSLTNMPDEGVMVTKFYFQEDE

SEQ ID NO: 14 - DNA, 456 bp, Artificial Sequence
  The sequence is synthesized.
cgaccctctgggagaaaatccagcaagatgcaagccttcacaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtaatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgag

SEQ ID NO: 15 - protein, 152 aa, Artificial Sequence
  The sequence is synthesized.
RPSGTKSSKMQAFTIWDVNQKTFYLRNNQLVAGYLQGPNVNLEEKIDVVPIEPHALFLGIHGGKMCLSCVKSGNETRLQLEAVNITDLSENRKQDKRFAFIRSDSGPTTSFESAACPGWFLCTAMEADQPVSLTNMPDEGVMVTKFYFQEDE

SEQ ID NO: 16 - DNA, 456 bp, Artificial Sequence
  The sequence is synthesized.
cgaccctctgggacaaaatccagcaagatgcaagccttcacaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtaatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgag

SEQ ID NO: 17 - protein, 235 aa, Artificial Sequence
  The sequence is synthesized.
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 18 - protein, 232 aa, Artificial Sequence
  The sequence is synthesized.
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 232 aa, Artificial Sequence
  The sequence is synthesized.
EPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 228 aa, Artificial Sequence
  The sequence is synthesized.
ERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 21 - protein, 229 aa, Artificial Sequence
  The sequence is synthesized.
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 585 aa, Artificial Sequence [sequence omitted]

SEQ ID NO: 23 - protein, 396 aa, Artificial Sequence
  The sequence is synthesized.
RPSGRKSSKMQAFRIWDVNQKTFYLRNNQLVAGYLQGPNVNLEEKIDVVPIEPHALFLGIHGGKMCLSCVKSGNETRLQLEAVNITDLSENRKQDKRFAFIRSDSGPTTSFESAACPGWFLCTAMEADQPVSLTNMPDEGVMVTKFYFQEDEGGGGSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 396 aa, Artificial Sequence
  The sequence is synthesized.
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSRPSGRKSSKMQAFRIWDVNQKTFYLRNNQLVAGYLQGPNVNLEEKIDVVPIEPHALFLGIHGGKMCLSCVKSGNETRLQLEAVNITDLSENRKQDKRFAFIRSDSGPTTSFESAACPGWFLCTAMEADQPVSLTNMPDEGVMVTKFYFQEDE

(19) United States
(12) Patent Application Publication
(10) Pub. No.: US 2024/0018206 A1
(43) Pub. Date: Jan. 18, 2024

(54) AN INTERLEUKIN-1 RECEPTOR ANTAGONIST AND A FUSION PROTEIN CONTAINING THE SAME

(71) Applicant: BEIJING VDJBIO CO., LTD., Beijing (CN)

(72) Inventors: Ziqiang LI, Beijing (CN); Rong MA, Beijing (CN); Xinsheng TIAN, Beijing (CN); Yiping SUN, Beijing (CN); Yiren LIU, Beijing (CN); Shaokui LI, Beijing (CN)

(21) Appl. No.: 17/927,625

(22) PCT Filed: Jul. 9, 2020
(86) PCT No.: PCT/CN2020/100985

§ 371 Date: Nov. 23, 2022

(30) Foreign Application Priority Data
May 25, 2020 (CN) 202010450177.7

(51) Int. Cl. C07K 14/545; C07K 14/715; C07K 14/79; C07K 14/765

(52) U.S. Cl. CPC C07K 14/545; C07K 14/7151; C07K 14/79; C07K 14/765; C07K 2319/30; A61K 38/00

(57) ABSTRACT

Provided is an Interleukin-1 Receptor Antagonist (IL-1RN) protein or a variant or analogue thereof, and a fusion protein comprising the following three-parts: the Interleukin-1 Receptor Antagonist protein or a variant or analogue thereof, a domain for half-life extension and an optional tumor necrosis factor receptor 2, and the preparation method and use thereof. The above-mentioned protein mutant and fusion protein have the effects of extended half-life, improved affinity with interleukin-1 receptor and superior biological activity, and find use in the field of treatment and prevention of inflammatory-associated diseases.

### FIELD OF THE INVENTION

The invention belongs to the field of biomedicine, and relates to the use of an IL-1RN mutants and a protein containing the same in medical treatment.

### BACKGROUND OF THE INVENTION

Interleukin-1 (IL-1), which is mainly composed of two proteins-IL-1α and IL-1β, is generated from the body's response to inflammatory stimulation, and is involved in the pathogenesis of various autoimmune and auto-inflammatory diseases, including inflammatory bowel disease, rheumatoid arthritis, cryopyrin-associated periodic syndromes, etc. IL-1α and IL-1β have similar biological activities, and can bind to IL-1 receptor (IL-1R1) with the aid of IL-1 receptor accessory protein (IL-1RAcP) to transmit signals into cells. IL-1α is expressed relatively wide, mainly in epithelial cells, keratinocytes and endothelial cells, and typically functions locally. IL-1β is primarily generated by monocytes and macrophages, is secreted throughout the body and has circulated effects. Under normal circumstances, both IL-1α and IL-1β are expressed at low levels, the levels of transcription and translation are dependent on the induction, and the processing and secretion are dependent on regulation. Loss of such regulatory steps will lead to syndromes characterized by fever, rash and arthritis.

IL-1 receptor antagonist (Interleukin 1 receptor antagonist, IL-1RN) is a high-affinity competitor for IL-1α and IL-β. It is induced by different cytokines in different cells and is a proteinic cytokine receptor antagonist present in human body. IL-1RN is capable of binding to IL-1R1 tightly, thereby blocking the binding of IL-1α and IL-1β to the respective receptors and thus antagonizing various biological effects of IL-1, and thus it can be used clinically to treat inflammatory diseases, in which IL-1 is involved in the process of pathological changes, including rheumatoid arthritis, CAPS, atopic dermatitis, hidradenitis suppurativa, etc.

Due to its small molecular weight, the short half-life (2-3 hours) of IL-1RN in plasma, it is often necessary to inject once a day to maintain the effective blood concentration in the body. Besides, in order to achieve significant clinical effects, the clinical dosage of IL-1RN is very high, e.g. 75-150 mg injected each time for rheumatoid arthritis. Excessively frequent high-dose injections will increase patient suffering and are prone to adverse reactions. By modifying IL-1RN, the present invention improves its biological activity and extends its half-life, thereby reducing the injection frequency and dosage, and maintaining a good clinical effect.

### SUMMARY OF THE INVENTION

### Definitions and Terms

Unless otherwise stated, the following definitions apply throughout of the present invention. Undefined terms can be understood according to the agreed definitions in the art.

Throughout the specification, unless the context requires otherwise, the word “comprise”, or variations, such as “comprises” or “comprising”, will be understood to mean including the elements or groups of the member or is an integer, but the elements or groups of any other members or other integers are not excluded.

As used in the specification and claims, the singular forms “a”, “an” and “the” include plural referents, unless the context clearly dictates otherwise. For example, the term “a cell” involves multiple cells, including mixtures thereof.

The term “about” or “approximately” means within an acceptable error range for a particular value as determined by one of ordinary skill in the art, which will depend in part on how the value is measured or determined, that is, the limitations of the measurement system. For example, the wording “about” means within the standard deviation of 1 or greater than 1 according to practice in the art. Alternatively, “about” means in the range of at most 20%, at most 10%, at most 5%, or at most 1% of a given value. Alternatively, especially for biological systems or processes, this term means within the order of magnitude of a value, preferably within 5 times, and more preferably within 2 times. In the case where a specific value is described in the application and claims, it should be assumed that the term “about” means within an acceptable error range for the specific value, unless otherwise stated.

The terms “polynucleotide” and “nucleic acid molecule” as used herein are used interchangeably, which means a polymeric form of nucleotides of any length. Polynucleotides may include deoxyribonucleotides, ribonucleotides, and/or analogs thereof. Nucleotides can have any three-dimensional structure and can perform any known or unknown functions. The term “polynucleotide” includes, for example, single-stranded, double-stranded and triple-helix molecules, genes or gene fragments, exons, introns, mRNA, tRNA, rRNA, ribozymes, antisense molecule, cDNA, recombinant polynucleotide, branched polynucleotide, aptamer, plasmid, vector, isolated DNA of any sequence, isolated RNA of any sequence, nucleic acid probes, and primers. Nucleic acid molecules can also include modified nucleic acid molecules (e.g., including modified bases with sugars, and/or internucleotide linkers).

The term “amino acid” is understood to include 20 kinds of naturally occurring amino acids; post-translationally in vivo modified amino acids, including but not limited to hydroxyproline, phosphoserine and phosphothreonine; and other unusual amino acids, including but not limited to 2-aminoadipic acid: hydroxylysine isododecane, norvaline, norleucine and ornithine. Moreover, the term “amino acid” includes D- and L-amino acids. Given below are further detailed descriptions of possible amino acids that can be used according to the invention and examples of unnatural amino acids.

The term “variant” refers to a peptide or polynucleotide that differs from a reference peptide or polynucleotide but retains the main properties. A typical peptide variant differs from another reference peptide in amino acid sequence. In general, the difference is limited so that the sequences of the reference peptide and the variant are similar overall and identical in many regions. The difference of the amino acid sequences of the variant and the reference peptide can be generated by one or more modifications (e.g., substitutions, additions and/or deletions). Peptide variants include conservatively modified variants (e.g., conservative variants having about 75%, about 80%, about 85%, about 90%, about 95%, about 98%, about 99% of the original sequence). The substituted or inserted amino acid residue may or may not be an amino acid residue encoded by the genetic codes. The peptide variant may be naturally occurring, such as an allelic variant, or it may be an unknown naturally occurring variant.

The term “analog” refers to protein modifications, including but not limited to methylation, acetylation, phosphorylation, adenylation, ubiquitination, ADP ribosylation, etc.; conjugates, including but not limited to antibody conjugates, polypeptide conjugates, etc.; conjugates, including but not limited to drug conjugates, polymer conjugates, etc. The above is only an enumeration, and all analogs having similar overall function to proteins are within the claimed scope.

The term “polypeptide” means any polymer consisting essentially of any of 20 kinds of natural amino acids, regardless of their size. Although the term “protein” is often used to refer to relatively large proteins and “peptide” is often used to refer to small polypeptides, these terms are often used partially overlapping in this field. Unless otherwise noted, the term “polypeptide” generally refers to proteins, polypeptides, and peptides.

The term “vector” is a nucleic acid molecule having the ability to self-replicate in a host cell and accept foreign DNA. The vector has its own origin of replication and a unique recognition position for one or more restriction enzymes that can be used to insert foreign DNA, and usually has a selectable marker (for example, a gene encoding for antibiotic resistance), and often has a recognition sequence for expressing the inserted DNA (for example, a promoter). Common vectors include plasmid vectors and phage vectors.

The term “cell” is intended to encompass any prokaryotic cell, eukaryotic cell, primary cell, or immortalized cell line, and any population of such cells in a tissue or organ. Preferably, the cells are derived from mammals (especially human) and can be infected by one or more pathogens. The “host cell” according to the present invention can be transfected, transformed, transduced or infected cells of any source, including prokaryotic cells, eukaryotic cells, mammalian cells, avian cells, insect cells, plant cells or bacterial cells, or it can be any source of cells useful for propagating the nucleic acids described herein.

The protein of the present invention or a variant or analogue thereof may have altered biological effects on different cell types, including but not limited to human primary cells, lymphocytes, red blood cells, retinal cells, liver cells, neurons, keratinocytes, endothelial cells, endoderm cells, ectoderm cells, mesoderm cells, epithelial cells, kidney cells, hepatocytes, bone cells, bone marrow cells, lymph node cells, dermal cells, fibroblasts, T cells, B cells, plasma cells, natural killer cells, macrophages, granulocytes, neutrophils, Langerhans cells, dendritic cells, eosinophils, basophils, breast cells, lobular cells, prostate cells, lungs cells, esophageal cells, pancreatic cells, β-cell (insulin-secreting cells), hemangioblasts, muscle cells, oval cells (hepatocytes), mesenchymal cells, brain microvascular endothelial cells, astrocytes, various populations including adult stem cells and embryonic stem cells, various progenitor cells; and other human immortalized transformation or cancer cell lines.

The percentage of homology is analyzed by software known in the art, for example, as determined by GAP (Needleman and Wunsh, 1970) analysis (GCG program), with the parameters: gap creation penalty=5, gap extension penalty=0.3. When the analyzed sequence is at least 15 amino acids in length, GAP analysis is performed on the at least 15 amino acid region of the two sequences involved in the test. More preferably, when the analyzed sequence is at least 50 amino acids in length, GAP analysis is performed on the at least 50 amino acid region of the two sequences involved in the test. More preferably, when the analyzed sequence is at least 100 amino acids in length, GAP analysis is performed on the at least 100 amino acid region of the two sequences involved in the test. More preferably, when the analyzed sequence is at least 250 amino acids in length, GAP analysis is performed on the at least 250 amino acid region of the two sequences involved in the test. Even more preferably, when the analyzed sequence is at least 500 amino acids in length, GAP analysis is performed on the at least 500 amino acid region of the two sequences involved in the test.

As used herein, a “variant” of IL-1RN refers to the amino acid sequence wherein one or more amino acids have been changed. The variant may have “conservative” changes, where the substituted amino acids have similar structural or chemical properties, for example, substitution of leucine with isoleucine. Alternatively, the variant may have “non-conservative” changes, such as glycine substituted by tryptophan. Similar minor changes can also include amino acid deletions or insertions, or both. Using computer programs well known in the art, such as DNAstar software, one can find a guide to determine which amino acid residues can be substituted, inserted or deleted without destroying biological or immunological activity.

The vector used in the present invention may be, for example, a phage, plasmid, cosmid, mini-chromosome, viral or retroviral vector. A vector useful for cloning and/or expressing the polynucleotide of the invention is a vector that can replicate and/or express the polynucleotide in a host cell that needs to replicate and/or express the polynucleotide. In general, polynucleotides and/or vectors can be used in any eukaryotic or prokaryotic cell, including mammalian cells (such as human cells (such as HeLa), monkey cells (such as Cos), rabbit cells (such as rabbit reticulocytes), rat cells, Hamster cells (such as CHO, NSO and baby hamster kidney cells) or mouse cells (such as L cells), plant cells, yeast cells, insect cells or bacterial cells (such as E. coli). As for examples of suitable vectors applicable for various types of host cells, for example, F. Ausubel et al. Current Protocols in Molecular Biology. Greene Publishing Associates and Wiley-Interscience (1992) and Sambrook et al. (1989) may be referred to. Host cells containing these polynucleotides may be used to express large amounts of proteins useful in, for example, drugs, diagnostic reagents, vaccines, and therapeutic agents. Various methods have been developed for operably linking polynucleotides to vectors via complementary sticky ends. For example, a complementary homopolymer sequence fragment can be added to the DNA segment intended to be inserted into the vector. The vector and the DNA segment are then linked by hydrogen bonds between the complementary homopolymer tails to form recombinant DNA molecules.

### BRIEF DESCRIPTION OF THE INVENTION

The present invention relates to an interleukin-1 receptor antagonist (IL-1RN) protein or a variant or analogue thereof, which has the sequence that maintains at least 70%, 75%, 80%, 85%, 90%, 95%, 96%, 97%, 98%, 99%, 99.5%, or 100% sequence identity to SEQ ID NO: 1. Further provided is a nucleotide sequence encoding the said protein, which has the sequence that maintains at least 70%, 75%, 80%, 85%, 90%, 95%, 96%, 97%, 98%, 99%, 99.5%, or 100% sequence identity to SEQ ID NO: 2, or hybridizes to the complementary sequence of one of the said nucleotide sequences under stringent hybridization conditions. The sequence shown in SEQ ID NO: 2 is the nucleotide sequence of a wild-type interleukin-1 receptor antagonist.

Further, the present invention provides a protein sequence of the interleukin-1 receptor antagonist (IL-1RN) protein or a variant or analogue thereof, which has at least 70%, 75%, 80%, 85%, 90%, 95%, 96%, 97%, 98%, 99%, 99.5%, or 100% sequence identity to one of the protein sequences set forth in SEQ ID NO: 3, 5, 7, 9, 11, 13 or 15, wherein the protein sequence comprises at least one of mutational sites selected from R5T, R14T, and D74N.

The present invention also provides a nucleotide sequence encoding the interleukin-1 receptor antagonist (IL-1RN) protein or a variant or analogue thereof, which has the sequence that has at least 70%, 75%, 80%, 85%, 90%, 95%, 96%, 97%, 98%, 99%, 99.5%, and 100% sequence identity to one of the nucleotide sequences set forth in SEQ ID NO: 4, 6, 8, 10, 12, 14, or 16, or hybridizes to the complementary sequence of one of the nucleotide sequences set forth in SEQ ID NO: 4, 6, 8, 10, 12, 14, or 16 under stringent hybridization conditions, wherein the nucleotide sequence comprises one or more nucleotide mutational sites which result in at least one of the mutational site of R5T, R14T, D74N in the encoded protein.

Further, the present invention provides a fusion protein, which comprises a domain for extending the half-life of the IL-1RN protein, and is generated from linking with the above-mentioned IL-1RN protein or a variant or analogue thereof inhibiting IL-1 activity; further, the fusion protein or a variant or analogue thereof may or may not comprise tumor necrosis factor receptor 2 (TNFR2) or a fragment or mutant thereof as a dual-target design. Preferably, the fragment of tumor necrosis factor receptor 2 (TNFR2) is the extracellular domain of the tumor necrosis factor receptor 2 (TNFR2).

In the context of the invention, the domain for extending the half-life of the IL-1RN protein includes but not limited to, one or more Fc domains selected from IgG1Fc, IgG2Fc, IgG3Fc, IgG4Fc, IgM, IgA, IgD; or serum albumin; or transferrin (Tf). In the context of the invention, Fc, skeleton region, or serum albumin is derived from primate mammals selected from the group consisting of humans, orangutans and gorillas, domestic animals (e.g. cattle, sheep, pigs, horses, donkeys), in-house laboratory animals (e.g. mice, rats, guinea pigs, hamsters, rabbits, companion animals (e.g. cats, dogs) and captured wild animals (e.g. rodents, foxes, deer, giraffes).

In the context of the invention, the sequence of TNFR2 fragment has at least 70%, 75%, 80%, 85%, 90%, 95%, 96%, 97%, 98%, 99%, 99.5% or 100% sequence identity to the sequence set forth in SEQ ID NO: 17, and may comprise mutations that do not affect its overall function.

In the context of the invention, the Fc domain may have a mutation site, including but not limited to the sequence that has at least 70%, 75%, 80%, 85%, 90%, 95%, 96%, 97%, 98%, 99%, 99.5% or 100% sequence identity to the sequence set forth in one of SEQ ID NOs: 18-21.

In the context of the invention, the sequence of serum albumin, includes but not limited to the sequence that has at least 70%, 75%, 80%, 85%, 90%, 95%, 96%, 97%, 98%, 99%, 99.5% and 100% sequence identity to the sequence set forth in SEQ ID NO: 22.

In the context of the invention, the domain for extending the half-life of the IL-1RN protein is linked to the IL-1RN protein inhibiting the activity of IL-1 via a flexible linker of general formula (GnS)m, wherein n is an integer of 0-6, preferably n is 0, 1, 2, 3, 4, 5 or 6, m is an integer of 1-4, preferably m is 1, 2, 3 or 4; preferably, the general formula is (GlyGlyGlyGlySer)m, wherein m is an integer of 1-3, preferably m is 1, 2, or 3. The general formula is only exemplary, and all the linker peptides that are capable of linking the said two parts are within the claimed scope.

Further, the present invention also provides a fusion protein or a variant or analogue thereof, including but not limited to the sequence that has at least 70%, 75%, 80%, 85%, 90%, 95%, 96%, 97%, 98%, 99%, 99.5%, or 100% sequence identity to the sequence set forth in SEQ ID NO: 23 or 24 (IL-1RN D74N-hIgG4 Fc S228P, hIgG4 Fc S228P-IL-1RN D74N). The sequences set forth in SEQ ID NOs: 23 and 24 are merely examples of fusion proteins. As mentioned above, all of the fusion proteins having or not having linkers, optionally, the domain for half-life extension, and optionally, dual target design are within the claimed scope. Furthermore, the present invention provides a nucleotide sequence encoding the said fusion protein.

Further, the said protein or a variant or analogue thereof, fusion protein or a variant or analogue thereof, as a protein unit, can be optionally combined into a polypeptide complex, and each unit may be the same or different, wherein the polypeptide complex has at least two protein units, and the protein units may be linked to each other via a linker peptide or otherwise.

The said protein or a variant or analogue thereof, fusion protein or a variant or analogue thereof, or polypeptide complex, optionally with N-terminal signal peptide known in the art, could be cloned and expressed in vectors known in the art, and optionally implanted into host cells.

And further, a pharmaceutical composition may be prepared, wherein the pharmaceutical composition is optionally mixed with one or more pharmaceutically acceptable carriers or excipients to formulate a pharmaceutical dosage form for different administration routes, including but not limited to such as tablet, capsule, powder, granule, syrup, solution, oral liquid, spiritus, tinctures, aerosols, dry powder inhalations, injections, sterile powders for injection, suppositories, etc. The protein or a variant or analogue thereof, fusion protein or a variant or analogue thereof, or polypeptide complex of the present invention can be administered via oral, intravenous, intramuscular, subcutaneous and other routes. “Pharmaceutically acceptable” ingredients are substances that are suitable for humans and/or animals without excessive adverse side effects (such as toxicity, irritation, and allergies), that is, with a reasonable benefit/risk ratio. “Pharmaceutically acceptable carrier” is a pharmaceutically or food acceptable solvent, suspension or excipient for delivering protein or a variant or analogue thereof, fusion protein or a variant or analogue thereof, or polypeptide complex of the invention to animals or humans. The carrier could be liquid or solid.

The present invention further provides a method for preparing an interleukin-1 receptor antagonist mutant and a fusion protein containing the same. The host cells transformed with the nucleotide sequence encoding the fusion protein could be cultured under the conditions suitable for expression and recovery of the protein from the cell culture, and the protein produced by the transformed cells could be secreted from or contained in the cells, depending on the sequence and/or vector used. With the three-step purification process conditions, the recombinant fusion protein expressed by CHO cells is purified, the overall protein yield is greater than 50%, and the purity of the desired protein is greater than 98.5% by HPLC.

Further, the desired protein designed, constructed and purified by the present invention is determined by an activity test. The results show that the mutant fusion protein has a three-fold increase in activity compared to the wild-type, and the addition of a linker peptide results in a further three-fold increase. Moreover, the mutant shows a complete inhibitory effect on the inflammatory response in the mouse model of CIA disease.

The IL-1RN mutant and fusion protein disclosed in the invention is capable of being used to prepare a medicament for the treatment of inflammatory diseases, including one or more of arthritis, enteritis, asthma, pulmonary fibrosis, glomerulonephritis, graft-versus-host reaction, acute lung injury, severe corneal burn, rheumatoid arthritis, cryopyrin-associated periodic syndromes (CAPS), atopic dermatitis, hidradenitis suppurativa, cardiovascular disease, non-small cell lung cancer, etc.

### BRIEF DESCRIPTION OF FIGURES

FIG. 1 is a Mabselect SuRe chromatogram according to an embodiment of the invention.

FIG. 2 is a purity graph of a Mabselect SuRe chromatography-eluted protein measured by SEC-HPLC according to an embodiment of the invention.

FIG. 3 is a Capto Phenyl (HS) hydrophobic chromatogram according to an embodiment of the invention.

FIG. 4 is a purity graph of a Capto Phenyl (HS) hydrophobic chromatography-eluted protein measured by SEC-HPLC according to an embodiment of the invention.

FIG. 5 is a Capto adhere complex ion exchange chromatogram according to an embodiment of the invention.

FIG. 6 is a purity graph of Capto adhere complex ion exchange chromatography flow-through protein measured by SEC-HPLC according to an embodiment of the invention.

FIG. 7 is a graph showing the electrophoresis (SDS-PAGE) results of samples for each purification step according to an embodiment of the invention. The samples of each lane are as follows:
1. Marker
2. Cell culture supernatant
3. Affinity chromatography-eluted protein solution
4. Hydrophobic chromatography-eluted protein solution
5. Complex chromatography flow-through protein solution

FIG. 8 is a graph showing the results of cell biological activity assay of IL-1RN-Fc fusion protein.

FIG. 9 is a graph showing the results of cell biological activity test of the dual-target fusion protein.

FIG. 10 is a graph showing the CIA efficacy results in DBA/1 mice.

### EMBODIMENTS OF THE INVENTION

Preparation of TNFR2-Fc Fusion Protein

According to the amino acid sequence of etanercept (TNFR2-FC) published in CN1829739(A), the nucleotide sequence is designed and the codons are optimized. The synthetic TNFR2-FC gene is cloned into vector pEE12.4. The constructed recombinant plasmid is sequenced and confirmed to be identical with the designed sequence. The recombinant plasmid is linearized with Pvu I and CHO-K1 cells are transfected with it. The CHO cell lines stably expressing the target protein are screened by MSX (L-methionine sulfoximine). The screened cell lines are cultured in shake flask. After fermentation, the cells and cell fragments in the fermentation broth are removed by centrifugation, and then filtered through 0.22 μM filter membrane to obtain the clear fermentation liquid. The fermentation broth is extracted by protein A affinity chromatography using MabSelect SuRe™ (GE Healthcare) as packing material. The packing is balanced with binding buffer (20 mM PB, 0.15 M NaCl, pH 7.2). After sample loading, the sample is washed to the baseline. Finally, the target protein is eluted with eluent buffer (50 mM sodium citrate, pH 3.3). The affinity chromatography eluent is concentrated and further purified by gel filtration chromatography using Superdex 200 (GE Healthcare) as packing material and PBS as the buffer. The elution peaks of gel filtration chromatography are collected for non-reductive SDS-PAGE analysis, and the elution peak with high electrophoretic purity and having molecular weight identical to TNFR2-Fc is selected for biological activity analysis, and TNFR2-Fc fusion protein is prepared.

Construction of Protein

First, the nucleotide sequence is codon optimized according to the amino acid sequence of the desired protein, and sent to the gene synthesis company for DNA synthesis, and then the target gene synthesized and sequenced correctly is inserted into the pEE12.4 vector. This vector contains the strong promoter hCMV-MIE, the replicon pEE6 ori, the terminator SV40 poly(A) signal, the origin of replication SV40(ori), the HindIII/EcoRI cleavage site, and the glutamine synthetase gene. The constructed recombinant plasmid is sequenced to confirm that the inserted sequence is completely consistent with the designed sequence. The recombinant plasmid is linearized with restriction endonuclease Pvu I, and then transfected into CHO-K1 cells. The CHO cell strains stably expressing the desired protein are selected using MSX (L-Methionine Sulfoximine).

Construction of Cell Strain

CHO-K1 cells are prepared in advance, and the cell density is adjusted to 0.5×10⁶ cells/mL the day before transfection. 1.43×10⁷ cells are taken, washed and centrifuged to remove GlutaMAX in the medium. The cells are suspended with 0.7 mL CD CHO medium, 40 μg of digested linear plasmid is added, mixed well, and transferred to a 0.4 cm electric cup. The electroporator is adjusted to a capacitance of 1000 μF and a voltage of 300V. After the electric shock, the cells are quickly transferred to 150 mL medium preheated at 37° C. in 50 μL/well, inoculated in 96-well plate, and static cultured in 5% CO₂ at 37° C. 24 hours after transfection, additional 150 μL of CD CHO medium (containing 66.6 μM MSX) is added to each well to achieve the final concentration of 50 μM MSX, and the culture is continued in 5% CO₂ at 37° C. Culturing for 21-28 days after transfection, picking out the grown monoclones, and after propagating culture, harvesting the supernatant for SDS-PAGE reduction electrophoresis detection. The density of protein band staining is positively correlated with protein concentration.

Purification of Desired Protein

The purification method of the interleukin-1 receptor antagonist fusion protein in this embodiment includes the following steps.

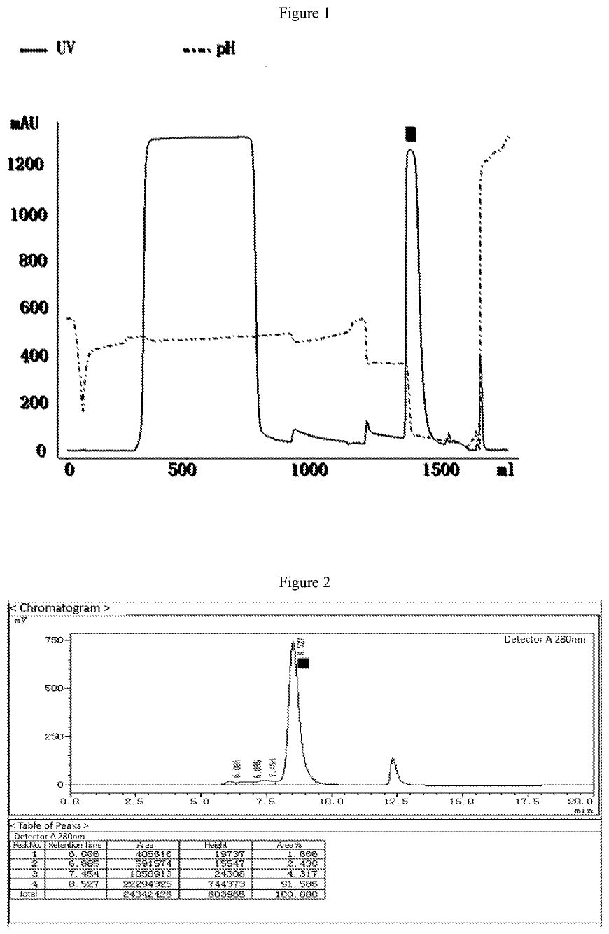

1. After Deep Filtration, the Obtained CHO Cell Culture is Firstly Purified by Mabselect SuRe Affinity Chromatography to Capture the Fusion Protein Solution.
1.1 Equilibrating with four column volumes of equilibration solution (20 mmol/L PB, 0.15 mol/L NaCl pH7.2), and the pH and conductivity monitoring values were consistent with the equilibration buffer.
1.2 Before loading the sample, the UV absorption value is set to zero. The culture supernatant is directly loaded after the deep filtration, the sample retention time is 9 minutes, and the loading volume is 15 mg/ml.
1.3 After loading the sample, washing with three column volumes of the affinity chromatography equilibration solution, then washing with wash solution 1 (20 mmol/L PB, 1.5 mol/L NaCl, 2.0 mol/L urea, pH 7.2).
1.4 The column is washed with washing solution 2 (100 mmol/L citric acid, 0.3 mol/L glycine, 10% sorbitol, pH 5.5). The displayed monitoring values of pH and conductivity are consistent with those of washing solution 2.
1.5 The eluent (100 mmol/L citrate, 0.3 mol/L glycine, 15% trehalose, 30% sorbitol, pH 3.7) is used to elute the sample, and the main peak of the protein is collected at 280 nm UV. The initial collection is at 120 mAU of UV, and the final collection is at 120 mAU of UV.
1.6 Washing with three column volumes of sodium hydroxide, then equilibrating the pH of the column to neutral stability using the equilibration solution, and then storing the column using three column volumes of ethanol.
1.7 As shown in FIG. 1, the black square mark shows the Mabselect SuRe chromatography image of the desired protein; as shown in FIG. 2, the black square mark shows the purity of the desired protein by HPLC.

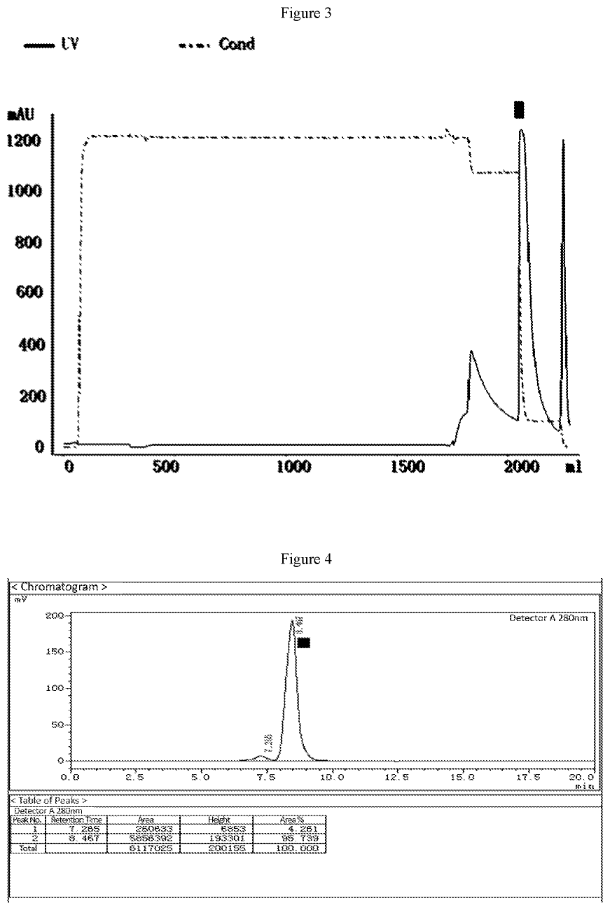

2. The Fusion Protein Captured in Step 1 is Purified by Capto Phenyl (HS) Hydrophobic Chromatography to Remove Most Impurities:
2.1 Sample pretreatment: The affinity eluted protein solution is diluted with a sample diluent (20 mmol/L PB, 3.0 mol/L NaCl, pH 7.1) at a volume ratio of 1:5 to adjust pH 7.1, and the conductivity is 165 to 175 mS/cm.
2.2 Using equilibration buffer (20 mmol/L PB, 2.2 mol/L NaCl pH7.1) to equilibrate the column with four column volumes. The monitoring values of pH and conductivity are consistent with those of the equilibration buffer.
2.3 Before loading the sample, setting the UV absorption value to zero. The sample is the protein solution eluted by Mabselect SuRe affinity chromatography in step 1, the sample retention time is 9 minutes, and the loading volume is 15 mg/ml.
2.4 After completion of loading the sample, washing the column with three column volumes of the equilibration solution to completely rinse off unbound components.
2.5 Further washing with two column volumes of washing buffer (20 mmol/L PB, 1.8 mol/L NaCl, pH7.1) to remove some weakly bound impurities.
2.6 Eluting the sample with elution buffer (20 mmol/L PB, 0.1 mol/L NaCl, pH7.1), collecting the main peak of the protein under 280 nm UV. Starting collecting at 100 mAU of UV, then stopping collecting at 100 mAU of UV.
2.7 Washing with three column volumes of regeneration buffer (0.1 mol/L NaOH), then washing with water to neutral, and storing the column in 20% of ethanol three column volumes.
2.8 As shown in FIG. 3, the black square mark shows the Capto Phenyl (HS) chromatogram of the desired protein; as shown in FIG. 4, the black square mark shows the purity graph of the desired protein by HPLC.

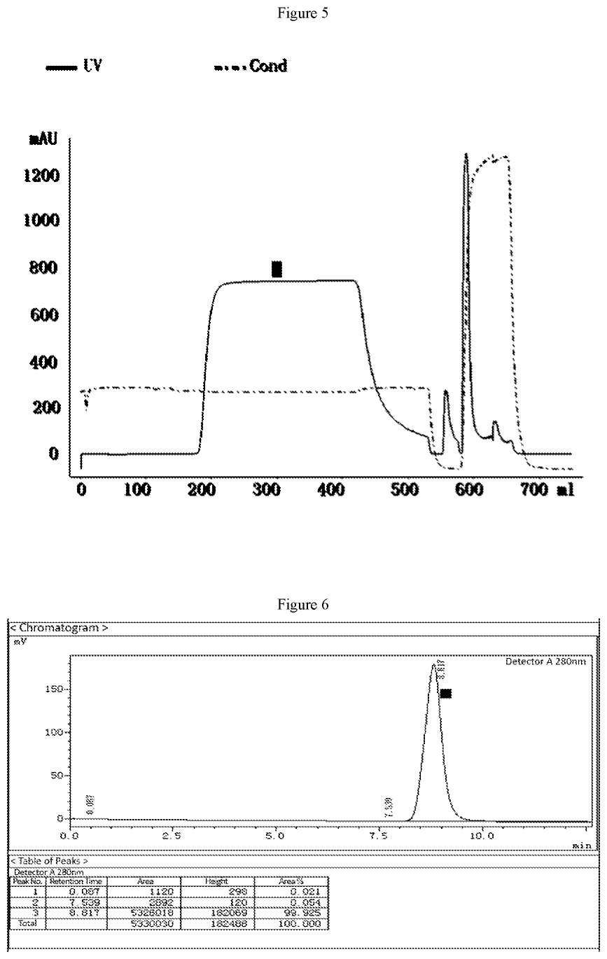

3. The Fusion Protein after Chromatography in Step 2 is Refined and Purified by Capto Adhere Complex Ion Exchange Chromatography to Remove Minor Impurities:
3.1 Use equilibration buffer (20 mmol/L PB, 0.5 mol/L NaCl pH5.9) to equilibrate the 5CV column. The monitoring values of pH and conductivity are consistent with those of the equilibration buffer.
3.2 Load the sample, which is the eluted protein solution in step 2. Start the loading after adjusting the sample to the same conditions as the equilibrium solution. The loading time is 4.5 minutes, and the loading volume is 115 mg/ml. After loading, equilibrate the sample with equilibration buffer until the sample is collected.
3.3 The main peak of the collected protein is at the UV absorption of 280 nm. The initial collection is at 80 mAU of UV, and the final collection is at 100 mAU of UV.
3.4 Regeneration: washing with 5 column volumes of 500 mmol/L NaOH and 2.0 mol/L NaCl, then washing to neutral with water, and storing the column in 20% ethanol of 3 column volumes.
3.5 As shown in FIG. 5, the black square mark shows the Capto adhere chromatogram of the desired protein; and as shown in FIG. 6, the black square mark shows the purity of the desired protein by HPLC.

4.0 Discussion

According to the three-step purification process conditions of Mabselect SuRe/Capto Phenyl(HS)/Capto adhere described above, the recombinant form of the fusion protein containing IL-1RN expressed by CHO cells is purified. The total yield of the protein is greater than 50%, and the purity of the desired protein is greater than 98.5% by SEC-HPLC.

Binding Affinity Assay

All SPR measurements are performed on a BIAcore 3000 instrument (GE Biosciences, Piscataway, N.J.). BIAcore software-BIAcore 3000 control software V3.2 is used to operate and control the BIAcore 3000 instrument. SPR data from the BIAcore 3000 instrument are analyzed using evaluation software V4.1, and the data are plotted using Graph Pad Prism software version 5. In the affinity assay, HBS-EP buffer (10 mM HEPES, 15 Mm NaCl, 3.4 nM EDTA, 0.005% P20) at 25° C. is used with the flow rate of 30 μL/min. The fusion protein shown is used as a ligand for constructing the reference channel of a chip. The analyte IL-1RN-Fc bound to the fixed receptor is measured, with a concentration of 1.2 to 100 nM (3-fold dilution). Each sample is injected at a flow rate of 30 μL/min for 3 minutes to bind to the fusion protein bound to the chip. Next, the binding buffer without analyte is passed through the chip at the same flow rate to dissociate the bound analyte. 500 s later, the regeneration solution (1M formic acid) is injected to remove the residual binding analyte. The data are analyzed using the Kinetics Guidelines and the manual fitting program attached to BiaEvaluation software V4.1.

TABLE 1
Binding activity of Typical Fusion Proteins

| | Recep- | Kₛ | K_{d} | K_{D} | Relative |
|---|---|---|---|---|---|
| Sample# | tor | (1/Ms) | (1/s) | (M) | Affinity |
| IL-1RN-Fc | IL-1R1 | 1.55E+05 | 1.82E−04 | 1.17E−9 | 1 |
| IL-1RN R5T-Fc | IL-1R1 | 1.72E+05 | 5.75E−05 | 3.34E−10 | 3.5 |
| IL-1RN R14T-Fc | IL-1R1 | 1.69E+05 | 8.05E−05 | 4.76E−10 | 2.5 |
| IL-1RN D74N-Fc | IL-1R1 | 1.88E+05 | 2.06E−05 | 1.10E−10 | 10.7 |
| IL-1RN R5T | IL-1R1 | 1.79E+05 | 4.65E−05 | 2.60E−10 | 4.5 |
| R14T-Fc | | | | | |
| IL-1RN R5T | IL-1R1 | 1.89E+05 | 1.83E−05 | 0.97E−10 | 12.1 |
| D74N-Fc | | | | | |
| IL-1RN R14T | IL-1R1 | 1.85E+05 | 1.98E−05 | 1.07E−10 | 11.0 |
| D74N-Fc | | | | | |
| IL-1RN R5T | IL-1R1 | 1.92E+05 | 1.75E−05 | 0.91E−10 | 12.9 |
| R14T D74N-Fc | | | | | |

(Exemplarily, hIgG4 Fc S228P is selected as an option of the domain for extending half-life, and exemplarily, GGGGSGGGGSGGGGS is selected as the linker peptide.)

Biological Activity Assay of the Recombinant Human IL-1RN-Fc Fusion Protein

The biological activity of said fusion protein is detected based on the principle that IL-1RN protein inhibits the process of IL-1β induced A375.s2 apoptosis. A375.s2 cells are cultured in MEM plus 10% FBS medium. A375.s2 cells are plated in a 96-well plate at 1.5×10⁵ cells/ml, 80 μl/well. Adjusting the concentration of IL-1β (R&D systems) to 10 μg/ml, and adding 10 μl per well. Then adjusting the concentration of IL-1RN-Fc fusion protein to 100 μg/ml, setting this as the highest concentration, and diluting to eight concentrations with 4-fold gradient, and adding 10 μl per well. After incubating at 37° C. for 96 hours, 20 μl of MTS detection reagent (Promega) is added to each well. After incubating at 37° C. for 0.5 hours, reading the microplate reader at 490 nm wavelength.

The test results are as follows:

TABLE 2
The results of biological activity assay of
recombinant human IL-1RN-Fc fusion protein

| | D74N | | D74N |
|---|---|---|---|
| IL-1RN-Fc | (w/o linker) | Wild-type | (w/linker) |
| IC₅₀(μg/ml) | 4.853 | 12.07 | 1.547 |

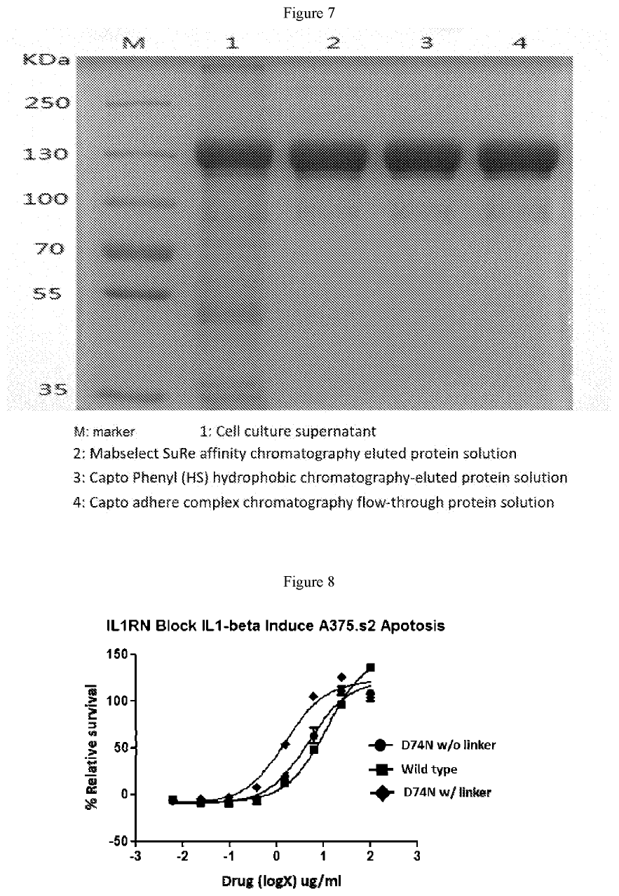

The results showed that, without linker, the mutant D74N (w/o linker) is three times the biological activity of the wild type. The biological activity of the mutant D74N (w/linker) is three times over the mutant D74N (w/o linker) without linker. See FIG. 8 for the results. (Exemplarily, IL-1RN (D74N) is selected as the mutant IL-1RN, exemplarily, hIgG4 Fc S228P is selected as the option of the domain for extending half-life, and exemplarily, GGGGSGGGGSGGGGS is selected as the linker.)

TABLE 3
The results of biological activity assay of recombinant
human IL-1RN mutant-Fc fusion protein

| | | | | | | | R5T |
|---|---|---|---|---|---|---|---|
| | | | | R5T | R5T | R14T | R14T |
| IL-1RN-Fc | R5T | R14T | D74N | R14T | D74N | D74N | D74N |
| IC₅₀(μg/ml) | 3.076 | 2.671 | 1.385 | 3.501 | 1.378 | 1.453 | 1.260 |

The results show that the biological activity of the single-site mutant is D74N>R14T>R5T; the biological activity of the three-site mutant is higher than that of the double-site and single-site mutants. (Exemplarily, hIgG4 Fc S228P is selected as the option of the domain for extending half-life, and exemplarily, GGGGSGGGGSGGGGS is selected as the linker.)

TABLE 4
The results of biological activity assay
of recombinant dual-target fusion protein

| | IL-1RN-Fc | TNFR2-Fc | IL-1RN-TNFR2-Fc |
|---|---|---|---|
| IL1RN activity | 0.333 | — | 0.714 |
| IC₅₀ (μg/ml) | | | |
| TNFR2 activity | — | 0.208 | 0.163 |
| IC₅₀ (μg/ml) | | | |

The results show that the biological activity of the dual-target fusion protein is comparable to that of the single-target fusion protein. See FIG. 9 for the results. (Exemplarily, IL1-RN (D74N) and TNFR2 are selected as dual target proteins, exemplarily, hIgG4 Fc S228P is selected as the option of the domain for extending half-life, and exemplarily, GGGGSGGGGSGGGGS is selected as the linker.)

Immunization Model and Treatment Experiment

1. Preparation of Mouse CIA Model

(1) Primary Immunization

3.3 ml of complete Freund's adjuvant is added into collagen in three times for emulsification. Each mouse is injected intraperitoneally with 0.4 ml of anesthesia. After the mice are anesthetized, the hair of the tail root is removed with an electric razor, and 100 μl of emulsified collagen is intradermally injected into the tail root.

(2) Booster Immunization

21 days after the initial immunization, the second booster immunization is performed, and the anesthesia and collagen for the second immunization of mice are prepared the day before the immunization. In the process of emulsifying the antigen, 3.3 ml of incomplete Freund's adjuvant is added into collagen in three times for emulsification. Each mouse is injected intraperitoneally with 0.4 ml of anesthesia. After the mice are anesthetized, the hair of the tail root is removed with an electric razor, and 50 μl of emulsified collagen is intradermally injected into the tail root.

2. Medical Treatment
(1) The mice start the onset of the disease 4 to 10 days after the second immunization. The modeled mice are randomly divided into groups within 24 hours of the onset of the disease, administered intraperitoneally, and observed continuously for 21 days. Joint scoring and weight measurement are performed every other day during the medical treatment.
(2) Experimental grouping

All mice are CIA modeled, except for the blank control group. The mice are scored and then randomized for treatment groups within 24 hours of the onset of the disease. The duration of medical treatment is 21 days.

TABLE 5
Mouse CIA model experiment

| | | Modeled | Frequency of | Dosage of | Mode of | Number/ |
|---|---|---|---|---|---|---|
| 1 | Group | or not | administration | administration | administration | Group |
| 2 | Blank control | No | — | — | — | 3 |
| 3 | PBS control | Yes | Once/week | PBS (400 μl) | Intraperitoneal | 5 |
| | | | | | injection | |
| 4 | TNFR2-Fc positive | Yes | Once/week | 10 mg/kg (400 μl) | Intraperitoneal | 8 |
| | control | | | | injection | |
| 5 | IL-IRN-Fc | Yes | Once/week | 10 mg/kg (400 μl) | Intraperitoneal | 8 |
| | | | | | injection | |
| | IL-IRN D74N-Fc | Yes | Once/week | 10 mg/kg (400 μl) | Intraperitoneal | 8 |
| | | | | | injection | |

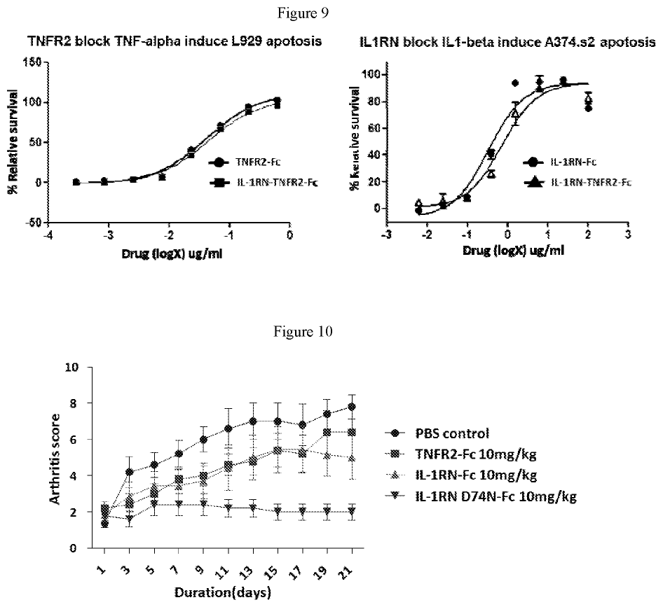

The results show that, in the mouse CIA animal model, the same injection dosage of wild-type IL-1RN-Fc shows the efficacy comparable to the known anti-rheumatic drug TNFR2-Fc, while the mutant IL-1RN D74N-Fc shows a complete inhibitory effect on the inflammatory response. See FIG. 10 for the results. (Exemplarily, the IL1-RN (D74N) mutant is selected, and GGGGSGGGGSGGGGS is selected as the linker, and Fc is hIgG4 Fc S228P.)

Although the present invention is described by specific embodiments, those skilled in the art should understand that various changes and equivalent substitutions can be made to the present invention without departing from the scope of the invention. In addition, the present invention can be variously modified for specific situations or materials without departing from the scope of the invention. Therefore, the present invention is not limited to the disclosure of the specific embodiments, but should include all embodiments falling within the scope of the claims of the invention.

1. An Interleukin-1 Receptor Antagonist (IL-1RN) protein or a variant or analogue thereof, characterized in that the protein or the variant or the analogue thereof has at least 70% identity to SEQ ID NO: 1 and includes at least one of mutated sites 5, 14, and 74 from N-terminus.

2. The protein or the variant or analogue thereof according to claim 1, wherein the mutated site is selected from at least one of R5T, R14T and D74N.

3. A nucleotide encoding the protein or the variant or analogue thereof according to claim 1.

4. A fusion protein or a variant or analogue thereof comprising a domain for extending the half-life of the IL-1RN protein, which is linked with the IL-1RN protein or the variant or analogue thereof according to claim 1.

5. The fusion protein or the variant or analogue thereof according to claim 4, further comprising tumor necrosis factor receptor 2 (TNFR2) or a fragment or variant thereof, preferably, the fragment is the extracellular domain of the tumor necrosis factor receptor 2 (TNFR2).

6. The fusion protein or the variant or analogue thereof according to claim 4, wherein the domain is selected from one or more of Fc domain, serum albumin and transferrin.

7. The fusion protein or the variant or analogue thereof according to claim 6, wherein the Fc domain is selected from one or more of IgG1Fc, IgG2Fc, IgG3Fc, IgG4Fc, IgMFc, IgAFc, IgDFc and a variant thereof.

8. The fusion protein or the variant or analogue thereof according to claim 4, wherein the domain for extending the half-life of the IL-1RN protein, the IL-1RN protein, and TNFR2 are linked by a linker.

9. The fusion protein or the variant or analogue thereof according to claim 8, wherein the linker has a general formula (GnS)m, where n is an integer of 0-6, preferably, n is 0, 1, 2, 3, 4, 5 or 6, m is an integer of 1-4, preferably, m is 1, 2, 3 or 4; preferably, the general formula is (GlyGlyGlyGlySer)m, where m is an integer of 1-3, preferably, m is 1, 2, or 3.

10. A vector that expresses the IL-1RN protein or the variant or analogue thereof according to claim 1.

11. A host cell comprising the vector according to claim 10.

12. A polypeptide complex comprising at least two of the fusion protein or the variant or analogue thereof according to claim 4.

13. The polypeptide complex according to claim 12, wherein the at least two of the protein or the variant or analogue thereof or the fusion protein or the variant or analogue thereof in each complex is identical or different.

14. A pharmaceutical composition comprising the protein or the variant or analogue thereof according to claim 1, and a pharmaceutically acceptable carrier or additive.

15. A method for treatment and prevention of an inflammatory-associated disease, comprising administrating a subject the pharmaceutical composition according to claim 14.

16. The method according to claim 15, wherein the inflammatory-associated disease is selected from one or more of arthritis, enteritis, asthma, pulmonary fibrosis, glomerulonephritis, graft-versus-host reaction, acute lung injury, severe corneal burn, rheumatoid arthritis, cryopyrin-associated periodic syndromes (CAPS), atopic dermatitis, hidradenitis suppurativa, cardiovascular disease, and non-small cell lung cancer.

17. A vector that expresses the fusion protein or the variant or analogue thereof according to claim 4.

18. A host cell comprising the vector according to claim 17.

19. A pharmaceutical composition comprising the fusion protein or the variant or analogue thereof according to claim 4, and a pharmaceutically acceptable carrier or additive.

20. A method for treatment and prevention of an inflammatory-associated disease, comprising administrating a subject the pharmaceutical composition according to claim 19.
